(19) United States
(12) Patent Application Publication
(10) Pub. No.: US 2019/0087727 A1
(43) Pub. Date: Mar. 21, 2019

(54) COURSE OF TREATMENT RECOMMENDATION SYSTEM

(71) Applicant: Navican Genomics, Inc., San Diego, CA (US)

(72) Inventor: John Scott Skellenger, San Diego, CA (US)

(21) Appl. No.: 16/133,595

(22) Filed: Sep. 17, 2018

Related U.S. Application Data
(60) Provisional application No. 62/560,128, filed on Sep. 18, 2017.

(51) Int. Cl. G06N 3/08; G16H 10/60; G06N 7/00

(52) U.S. Cl. CPC G06N 3/08; G06N 7/005; G16H 10/60

(57) ABSTRACT

A system for generating a course of treatment (“COT”) recommender for recommending COTs for patients using machine learning is provided. A machine learning treatment recommendation (“MLTR”) system trains a COT recommender using training data that includes a feature vector and a label for each patient in a group of patients. The features of the feature vector may include features derived from patient data. A label is a course of treatment for a patient referred to as a labeling course of treatment. The MLTR system generates the training data from patient data collected over time. The MLTR system then uses the training data to train the COT recommender using a machine learning technique. Once the COT recommender has been trained, the COT recommender can be applied to a feature vector of patient data of a patient to generate an MLTR recommended course of treatment for the patient.

### CROSS-REFERENCE TO RELATED APPLICATION

This application claims the benefit of U.S. Provisional Application No. 62/560,128, filed on Sep. 18, 2017, which is hereby incorporated by reference in its entirety.

### BACKGROUND

Genetics often plays a role in the development of cancers in patients. When a cancer patient is treated, genetic testing is performed so that the test results can inform the treatment. For example, if a patient has a certain genomic variant or combination of genomic variants (e.g., insertions, deletions, rearrangements, fusions, or other genomic anomalies) that are associated with the patient's cancer, the patient may be treated with drugs that are effective at treating patients with that specific mutation, called targeted therapies or immunotherapies. The following table lists examples of drugs that may be effective at treating specific mutations within a gene.

| Gene Containing Mutation | Drugs |
|---|---|
| MSH6 | Pembrolizumab and Nivolumab |
| PIK3CA | Everolimus and Temsirolimus |
| KRAS | Trametinib and Cobimetinib |

When treating a patient with mutations in genes MSH6, PIK3CA, and KRAS, a medical provider (e.g., oncologist) may decide to first treat the patient with one or more of the drugs associated with a mutation in gene PIK3CA. Depending on the results of the treatment, the medical provider may decide to continue treatment with the drug associated with a mutation in gene MSH6. Selecting the specific order of treatment is based on the discretion of the practicing physician and will vary based on the individual case. Based on the response to treatment, the medical provider may continue with alternative treatment options in combination with targeted therapies or alone (e.g., chemotherapy, surgery, or photon therapy). A course of treatment specifies both the treatments and the ordering of the treatments. A course of treatment may include a drug or combination of drugs (i.e., regimen) to be administered to a patient. A course of treatment may also specify a comprehensive treatment plan (e.g., chemotherapy, surgery, and proton therapy).

A vast amount of research is conducted in the field of cancer treatments by researchers at universities, hospitals, cancer research centers, drug companies, medical device manufacturers, and so on. Because these researchers publish such a large number of articles annually on treatments for cancers, it is difficult for a medical provider to not only read the articles but also assess the value of the treatments especially when articles indicate differences in the effectiveness of various treatments. As such, the clinical practice of medicine can become localized and anecdotal based on the predominant patient population or case load a practicing physician may encounter.

The National Comprehensive Cancer Network (“NCCN”) is an alliance of cancer centers that publishes guidelines or recommendations for treatments of various types of cancers. The NCCN guidelines and similar clinical pathway standards organizations' recommended courses of treatment are based on the evidence of treatments reported by articles relating to cancer research and are thus referred to as being evidence-based. For example, a recommended course of treatment for a patient may be based on research relating to the treatment of a cohort of patients. The recommended course of treatment for a patient with gene mutations in MSH6, PIK3CA, and KRAS may be to treat first with the drug Temsirolimus, followed by the drugs Nivolumab and Trametinib, depending on the efficacy of the prior drug. Unfortunately, the recommended courses of treatment may not be based on the most current clinical approvals because it can take time (e.g., a year or more) for the NCCN to update the recommended courses of treatment based on new evidence. As such, leveraging the most current treatments will be based on the knowledge and discretion of the practicing physician.

### BRIEF DESCRIPTION OF THE DRAWINGS

FIG. 1 is a flow diagram that illustrates overall processing of a machine learning treatment recommendation (MLTR) system in some embodiments.

FIG. 2 is a flow diagram that illustrates a process of collecting courses of treatment for a patient from various data sources for use by the MLTR system.

FIG. 3 is a block diagram illustrating components of the MLTR system in some embodiments.

FIG. 4 is a flow diagram that illustrates processing of a train COT recommender component in some embodiments.

FIG. 5 is a flow diagram that illustrates processing of a generate training data component of the MLTR system in some embodiments.

FIG. 6 is a flow diagram that illustrates the processing of a generate patient feature vector component of the MLTR system in some embodiments.

FIG. 7 is a flow diagram that illustrates processing of a generate recommended course of treatment component of the MLTR system in some embodiments.

### DETAILED DESCRIPTION

A method and system for generating a COT recommender for recommending courses of treatment (“COTs”) for patients using machine learning is provided. In some embodiments, a machine learning treatment recommendation (“MLTR”) system trains a COT recommender using training data that includes a feature vector and a label for each patient in a group of patients. The features of the feature vector may include features derived from patient data such as evidence-based recommended courses of treatments for patients, personal characteristic data (e.g., age, diagnoses, medical procedures, lab results, disease and supportive care therapies, previous test results, and so forth) for patients, medical history data for patients, diagnosis data for patients, and patient-reported outcomes (“PROs”). A label is a course of treatment for a patient, referred to as a labeling course of treatment. For example, a labeling course of treatment may be a recommended course of treatment developed for the patient by a panel of one or more experts, such as clinical experts, based on the evidence-based recommended course of treatment for the patient and personal characteristics, medical history, and diagnosis of the patient. Such a recommended course of treatment is referred to as a clinical expert panel recommended course of treatment. The MLTR system generates the training data from patient data collected over time. The MLTR system then uses the training data to train the COT recommender. For example, the COT recommender may be a neural network, and the training data is used to learn parameters (e.g., weights for activation functions) of hidden layers of the neural network. Once the COT recommender has been trained, the COT recommender can be applied to a feature vector of patient data of a patient to generate an MLTR recommended course of treatment for the patient.

Currently, various organizations (e.g., universities and companies) have clinical expert panels, such as a molecular tumor board, that review patient data for a patient and generate a clinical expert panel recommended course of treatment for the patient. When deciding on a course of treatment, the clinical experts typically take into consideration research that is more current than that used to generate the evidence-based recommended courses of treatment. Since the clinical experts of a clinical expert panel may weigh the same research differently, each may recommend a somewhat different course of treatment. A mediation process may be used to arrive at the final clinical expert panel recommended course of treatment. Although a clinical expert panel provides one recommended course of treatment for a patient, different clinical expert panels may recommend different courses of treatment for the same patient or cohort patients. Because the MLTR system trains the COT recommender based on clinical expert panel recommended courses of treatment and actual treatment decisions, the COT recommender is indirectly based on current evidence-based research.

In some embodiments, the MLTR system may update the COT recommender frequently to factor in the most recent clinical expert panel recommended courses of treatment. In addition, the MLTR system may also weigh more recent training data more heavily so that current trends in recommended courses of treatment can quickly be reflected in the MLTR recommended courses of treatment. The MLTR system may also factor in results of actual courses of treatment, which may deviate from the MLTR recommended courses of treatment, so that the COT recommender can factor in the positive, neutral (i.e., no change), and negative results of recommended courses of treatment.

The MLTR system may be trained to generate recommended courses of treatment that are similar to evidence-based recommended courses of treatment, clinical expert recommended courses of treatment of a certain clinical expert, clinical expert panel recommended courses of treatment, or actual courses of treatment. For example, to generate recommended courses of treatment that are similar to the evidence-based recommended courses of treatment, the MLTR system may label feature vectors of features derived from patient data with the evidence-based recommended courses of treatment. Once such a COT recommender is trained, it can be used to generate self-supporting, evidence-based courses of treatment without having to review, for example, the NCCN guidelines on a case-by-case basis. The features vectors would be labeled in a similar manner to generate recommended courses of treatment for the other recommended courses of treatment.

The MLTR system may employ different types of machine learning techniques to generate the COT recommender. For example, a neural network with a number of hidden layers may be trained using the training data. As another example, a Bayesian network may be trained using the training data to generate a probability for various courses of treatment or a probability for individual treatments. When trained to generate a probability for individual treatments, the feature vectors may include a feature indicating position of the treatment in a course of treatment and may be labeled with the treatment. As another example, a support vector machine for each possible treatment may be trained to classify whether that treatment should be included in the recommended course of treatment. In such a case, the feature vectors for patients include a feature indicating a treatment and are labeled, for example, to indicate whether that treatment was included in a course of treatment for that patient. To generate an MLTR recommended course of treatment for a patient, the support vector machine can be used to identify treatments to be considered. The treatments can then be ordered using, for example, a rule-based system. The treatments may also be ordered using a Bayesian network that is trained on the treatments and selected patient data and their desired orderings. The MLTR system may also use clustering techniques to generate clusters of patients with similar feature vectors. For each labeling course of treatment for the patients in a cluster, the MLTR system may generate a percentage to indicate what percentage of the patients in the cluster were labeled with the course of treatment. The MLTR system may recommend a course of treatment when its threshold percentage is above a certain level (e.g., 75%). The MLTR system may also factor in positive and negative results of actual courses of treatments. For example, support vector machines may be trained to classify each treatment as likely having a positive or negative result based on the feature vector for a patient.

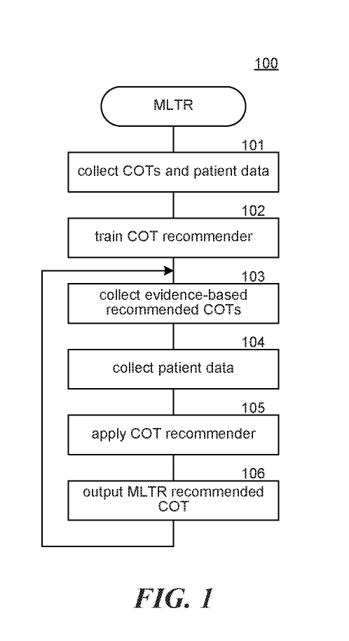

FIG. 1 is a flow diagram that illustrates overall processing of the MLTR system in some embodiments. The MLTR system 100 trains a COT recommender based on recommended courses of treatment and patient data for a group of patients and then uses the COT recommender to generate MLTR recommended courses of treatment for patients. In block 101, the MLTR system collects course of treatment data and patient data for the patients. In block 102, the MLTR system trains a COT recommender using training data derived from the collected courses of treatment and patient data. In blocks 103-106, the MLTR system uses the COT recommender to recommend courses of treatments for patients. In block 103, the MLTR system collects an evidence-based recommended course of treatment for a patient. In block 104, the MLTR system collects patient data for the patient. In block 105, the MLTR system applies the COT recommender to data derived from the evidence-based recommended course of treatment and the patient data for the patient to generate an MLTR recommended course of treatment. In block 106, the MLTR system provides the MLTR recommended course of treatment for use by a medical provider of the patient.

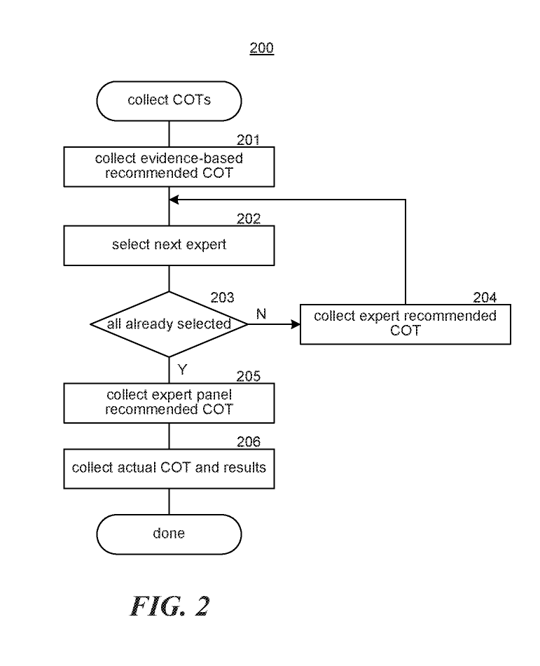

FIG. 2 is a flow diagram that illustrates a process of collecting courses of treatment for a patient from various data sources for use by the MLTR system. A collect courses of treatment process 200 may collect various recommended and actual courses of treatment. In block 201, the process collects an evidence-based recommended course of treatment for the patient generated based on, for example, guidelines of the NCCN. In blocks 202-204, the process loops collecting clinical expert recommended courses of treatment of the clinical experts of a clinical expert panel. Each clinical expert may have based their recommended course of treatment on the evidence-based recommended course of treatment and patient data for the patient. If the recommended courses of treatment by the clinical experts differ, then a mediation process may have been used to generate a final clinical expert panel recommended course of treatment. In block 205, the process collects the clinical expert panel recommended course of treatment. A medical provider for the patient may consider the evidence-based recommended course of treatment and the clinical expert panel recommended course of treatment along with patient data to decide upon an actual course of treatment. The actual course of treatment may be the same as the evidence-based recommended course of treatment or the clinical expert panel recommended course of treatment or may deviate from both recommended courses of treatment. The medical provider would typically record the results of the actual course of treatment. In block 206, the process collects the actual course of treatment along with its results. The process then completes.

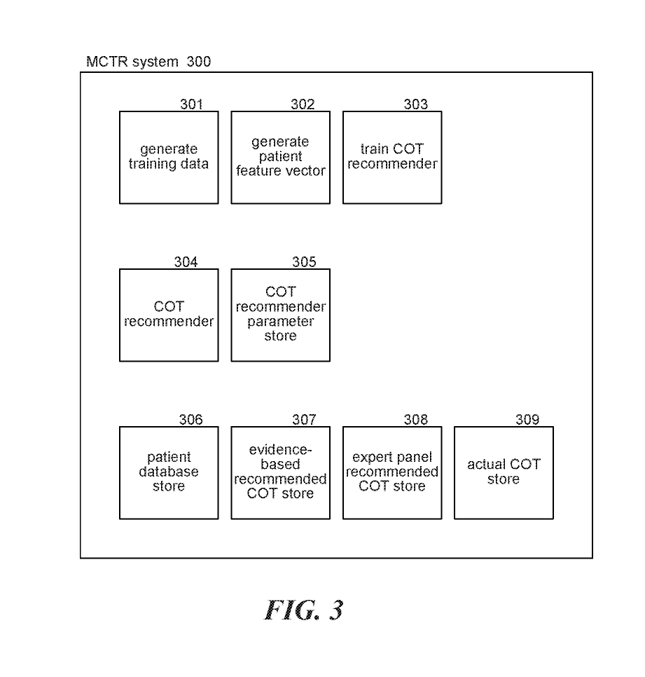

FIG. 3 is a block diagram illustrating components of the MLTR system in some embodiments. An MLTR system 300 includes a generate training data component 301, a generate patient feature vector component 302, a train COT recommender component 303, a COT recommender component 304, and a COT recommender parameter store 305. The generate training data component collects courses of treatments for patients and patient data, invokes the generate patient feature vector component to generate feature vectors for the patients, and labels the feature vectors. The train COT recommender component trains a COT recommender using the training data and stores the parameters for the COT recommender in the COT recommender parameter store. The COT recommender component inputs a feature vector for a patient and generates an MLTR recommended course of treatment based on the stored parameters. The MLTR system may also include a patient database store 306, an evidence-based recommended COT store 307, a clinical expert panel recommended COT store 308, and an actual COT store 309 that store data collected from various data sources.

The computing systems used by the MLTR system may include a central processing unit, input devices, output devices (e.g., display devices and speakers), storage devices (e.g., memory and disk drives), network interfaces, graphics processing units (e.g., to assist in machine learning), accelerometers, cellular radio link interfaces, global positioning system devices, and so on. The computing systems may include servers of a data center, massively parallel systems, and so on. The computing systems may access computer-readable media that include computer-readable storage media and data transmission media. The computer-readable storage media are tangible storage means that do not include a transitory, propagating signal. Examples of computer-readable storage media include memory such as primary memory, cache memory, and secondary memory (e.g., DVD, flash drive) and other storage. The computer-readable storage media may have recorded on them or may be encoded with computer-executable instructions or logic that implements the MLTR system. The data transmission media are used for transmitting data via transitory, propagating signals or carrier waves (e.g., electromagnetism) via a wired or wireless connection.

The MLTR system may be described in the general context of computer-executable instructions, such as program modules and components, executed by one or more computers, processors, or other devices. Generally, program modules or components include routines, programs, objects, data structures, and so on that perform tasks or implement data types. Typically, the functionality of the program modules may be combined or distributed as desired in various embodiments. Aspects of the MLTR system may be implemented in hardware using, for example, an application-specific integrated circuit (ASIC).

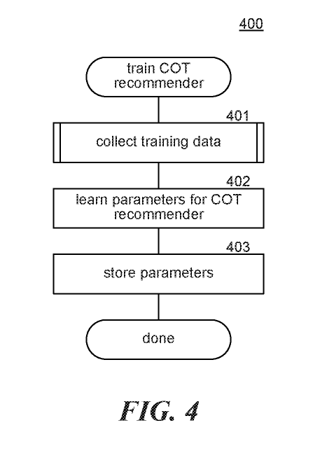

FIG. 4 is a flow diagram that illustrates processing of a train COT recommender component in some embodiments. A train COT recommender component 400 trains a COT recommender based on training data collected for a group of patients. In block 401, the component invokes a collect training data component to collect the training data. In block 402, the component learns the parameters for the COT recommender. In block 403, the component stores the learned parameters in the COT recommender parameter store and then completes.

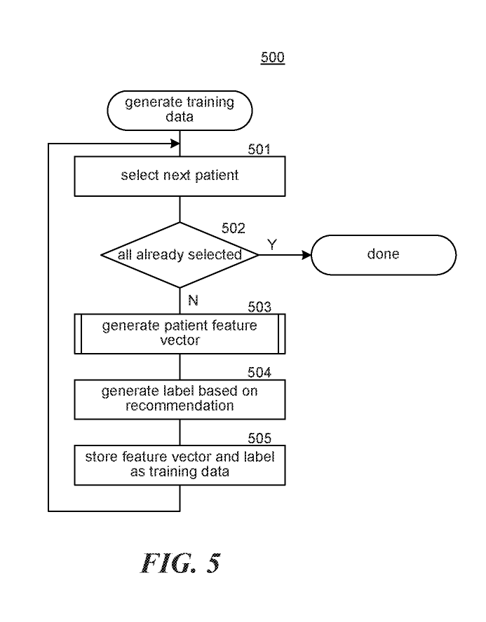

FIG. 5 is a flow diagram that illustrates processing of a generate training data component of the MLTR system in some embodiments. A generate training data component 500 is invoked to generate the training data for use in training a COT recommender. In block 501, the component selects the next patient in a group of patients. In decision block 502, if all the patients have already been selected, then the component completes, else the component continues at block 503. In block 503, the component invokes a generate patient feature vector component to generate a feature vector for the selected patient. In block 504, the component generates a label based on a recommended course of treatment for the patient. For example, the label may be a clinical expert panel recommended course of treatment for the patient. In block 505, the component stores the feature vector and label as training data and then loops to block 501 to select the next patient.

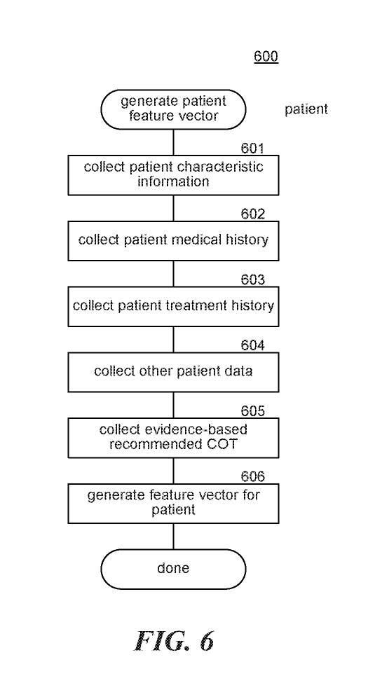

FIG. 6 is a flow diagram that illustrates the processing of a generate patient feature vector component of the MLTR system in some embodiments. A generate patient feature vector component 600 is invoked to collect data relating to the patient and generate a feature vector from the data. In block 601, the component collects patient characteristic information. In block 602, the component collects patient medical history information. In block 603, the component collects patient treatment history. In block 604, the component collects other patient data (e.g., regional history, occupation and education level). In block 605, the component collects an evidence-based recommended course of treatment for the patient. In block 606, the component generates a feature vector for the patient based on the collected data and then completes.

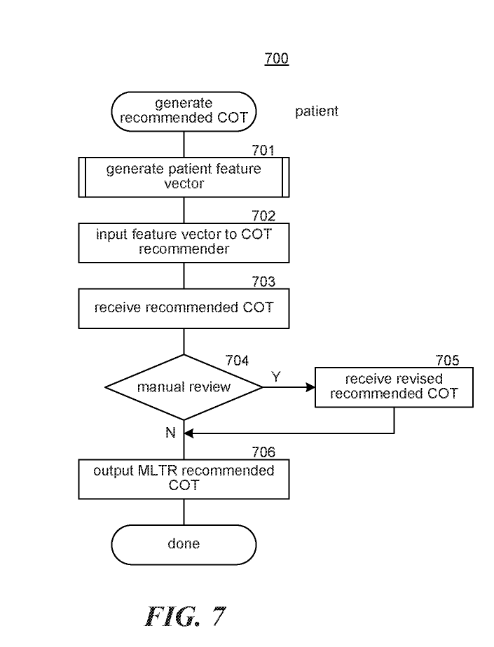

FIG. 7 is a flow diagram that illustrates processing of a generate recommended course of treatment component of the MLTR system in some embodiments. A generate recommended course of treatment component 700 is passed a patient profile and generates an MLTR recommended course of treatment for the patient. In block 701, the component invokes the generate patient feature vector component passing an indication of the patient to generate a feature vector for the patient. In block 702, the component inputs the feature vector to the COT recommender. In block 703, the component receives the recommended course of treatment from the COT recommender based on the input feature vector. In decision block 704, if a manual review is to be performed on the recommended course of treatment, then the component continues at block 705, else the component continues at block 706. In block 705, the component receives a revised recommended course of treatment. In block 706, the component outputs the MLTR recommended course of treatment and then completes.

Although the subject matter has been described in language specific to structural features and/or acts, it is to be understood that the subject matter defined in the appended claims is not necessarily limited to the specific features or acts described above. Rather, the specific features and acts described above are disclosed as example forms of implementing the claims. Accordingly, the invention is not limited except as by the appended claims.

I/We claim:

1. A method performed by a computing system for generating a recommender for recommending courses of treatment for patients, the method comprising:
for each of a plurality of patients,
accessing personal characteristic data for that patient;
accessing medical history data for that patient;
accessing an evidence-based recommended course of treatment for that patient;
generating a feature vector for the patient based on the personal characteristic data, the medical history data, and the evidence-based recommended course of treatment for that patient;
accessing a labeling course of treatment for that patient; and
labeling the feature vector with the labeling course of treatment for that patient to form a labeled feature vector; and
training the recommender, using the labeled feature vectors as training data, to generate a recommended course of treatment for a patient.

2. The method of claim 1 wherein the recommender is based on a neural network.

3. The method of claim 1 wherein the recommender is based on a Bayesian network.

4. The method of claim 1 where the recommender is based on a support vector machine.

5. The method of claim 1 wherein the recommender is based on clustering.

6. The method of claim 1 wherein the labeling course of treatment is a clinical expert panel recommended course of treatment for that patient.

7. The method of claim 1 wherein the labeling course of treatment is an actual course of treatment for that patient.

8. The method of claim 7 further comprising, for each of the plurality of patients, accessing a clinical expert panel recommended course of treatment for that patient and wherein the generating of the feature vector for that patient is further based on the clinical expert panel recommended course of treatment for that patient.

9. The method of claim 7 wherein the actual course of treatment for that patient is deemed to have had a positive, neutral, or negative result.

10. The method of claim 1 wherein a recommended course of treatment for a patient indicates, for mutations of that patient, a recommended ordering of treatments based on the mutations.

11. The method of claim 1 wherein a treatment of the recommended course of treatment specifies a recommended drug or drug combination or characterizes a comprehensive treatment plan to be administered to the patient.

12. The method of claim 1 wherein the recommender recommends multiple recommended courses of treatment for a patient along with a probability for each course of treatment.

13. The method of claim 12 wherein the probability indicates probability of an expected outcome.

14. The method of claim 1 wherein the personal characteristic data is selected from a group consisting of age, diagnoses, medical procedures, lab results, disease therapies, supportive case therapies, and test results.

15. The method of claim 1 wherein the medical history data for a patient includes immune markers, lab results, and/or treatment history.

16. The method of claim 1 further comprising accessing patient-reported outcomes for that patient and wherein the feature vector is further based on the patient-reported outcomes.
